(19) United States
(12) Patent Application Publication
(10) Pub. No.: US 2022/0378649 A1
(43) Pub. Date: Dec. 1, 2022

(54) METHODS, SYSTEMS, APPARATUSES, AND DEVICES FOR FACILITATING APPLICATION OF VARYING PRESSURE TO A BODY OF AN INDIVIDUAL

(71) Applicant: Shailen PATEL, London (GB)

(72) Inventor: Shailen PATEL, London (GB)

(21) Appl. No.: 17/795,114

(22) PCT Filed: Jan. 25, 2021
(86) PCT No.: PCT/IB2021/050566

§ 371 Date: Jul. 25, 2022

Related U.S. Application Data
(60) Provisional application No. 62/965,316, filed on Jan. 24, 2020.

(51) Int. Cl. A61H 21/00; A61H 9/00; A61N 1/05

(52) U.S. Cl. CPC A61H 21/00; A61H 9/0071; A61N 1/0548; A61H 9/0057; A61H 2201/5056; A61H 2201/1238; A61H 2201/0103; A61H 2205/02; A61H 2201/5005; A61H 2201/5071; A61H 2201/5012; A61H 2201/10; A61H 2201/1604

(57) ABSTRACT

Disclosed herein is an apparatus for facilitating application of varying pressure to a body of an individual, in accordance with some embodiments. Accordingly, the apparatus comprises pumps, actuators, and a valve. Further, the pumps are attached to the body using a coupling assembly. Further, the coupling assembly is configured to be attached to the body. Further, the coupling assembly comprises an interior cavity. Further, a first pump of the pumps is configured for drawing a first amount of air from the interior cavity. Further, a second pump of the pumps is configured for drawing a second amount of the air from the interior cavity and blowing a third amount of the air in the interior cavity. Further, the actuators are configured for actuating the pumps. Further, the valve is configured for allowing entering and exiting of an amount of the air from the interior cavity through the valve.

The current application is a Patent Cooperation Treaty (PCT) application and claims a priority to the U.S. provisional patent application Ser. No. 62/965,316 filed on Jan. 24, 2020.

### FIELD OF THE INVENTION

Generally, the present disclosure relates to the field of medical and laboratory equipment. More specifically, the present disclosure relates to methods, systems, apparatuses, and devices for facilitating application of varying pressure to a body of an individual.

### BACKGROUND OF THE INVENTION

Existing apparatuses for facilitating application of varying pressure to a body of an individual are deficient with regard to several aspects. For instance, current apparatuses do not apply varying pressure to the body of the individual.

Therefore, there is a need for improved methods, systems, apparatuses, and devices for facilitating application of varying pressure to a body of an individual that may overcome one or more of the above-mentioned problems and/or limitations.

### SUMMARY OF THE INVENTION

This summary is provided to introduce a selection of concepts in a simplified form, that are further described below in the Detailed Description. This summary is not intended to identify key features or essential features of the claimed subject matter. Nor is this summary intended to be used to limit the claimed subject matter's scope.

Disclosed herein is an apparatus for facilitating application of varying pressure to a body of an individual, in accordance with some embodiments. Accordingly, the apparatus may include a plurality of pumps, a plurality of actuators, and at least one valve. Further, the plurality of pumps may be configured to be fluidly coupled with the body of the individual using a coupling assembly. Further, the coupling assembly may be configured to be attached to the body. Further, the coupling assembly may include an interior cavity. Further, the interior cavity may be coupled with a body cavity of the individual. Further, the plurality of pumps may include a first pump and a second pump. Further, the first pump may be configured for drawing a first amount of air from the interior cavity for generating a reduced pressure of a first value in the body cavity. Further, the second pump may be configured for drawing at least one second amount of the air from the interior cavity and blowing at least one third amount of the air in the interior cavity for varying the reduced pressure in the body cavity between at least one second value and at least one third value from the first value. Further, the plurality of actuators may include a first actuator and a second actuator. Further, the first actuator may be operationally coupled with the first pump and the second actuator may be operationally coupled with the second pump. Further, the first actuator may be configured for actuating the first pump. Further, the second actuator may be configured for actuating the second pump. Further, the drawing of the first amount of the air from the interior cavity may be based on the actuating of the first pump. Further, the drawing of the at least one second amount of the air from the interior cavity and the blowing of the at least one third amount of the air in the interior cavity may be based on the actuating of the second pump. Further, the at least one valve may be fluidly coupled with the plurality of pumps. Further, the at least one valve may be configured for transitioning between a plurality of valve states based on the reduced pressure. Further, the at least one valve may be configured for allowing at least one of entering and exiting of at least one amount of the air from the interior cavity through the at least one valve based on the transitioning. Further, the allowing of the at least one of the entering and the exiting of the at least one amount of the air from the interior cavity maintains the reduced pressure within a predetermined value range of the reduced pressure.

Further disclosed herein is an apparatus for facilitating application of varying pressure to a body of an individual, in accordance with some embodiments. Accordingly, the apparatus may include a plurality of pumps, a plurality of actuators, at least one valve, at least one sensor, and a processing device. Further, the plurality of pumps may be configured to be fluidly coupled with the body of the individual using a coupling assembly. Further, the coupling assembly may be configured to be attached to the body. Further, the coupling assembly may include an interior cavity. Further, the interior cavity may be coupled with a body cavity of the individual. Further, the plurality of pumps may include a first pump and a second pump. Further, the first pump may be configured for drawing a first amount of air from the interior cavity for generating a reduced pressure of a first value in the body cavity. Further, the second pump may be configured for drawing at least one second amount of the air from the interior cavity and blowing at least one third amount of the air in the interior cavity for varying the reduced pressure in the body cavity between at least one second value and at least one third value from the first value. Further, the plurality of actuators may include a first actuator and a second actuator. Further, the first actuator may be operationally coupled with the first pump and the second actuator may be operationally coupled with the second pump. Further, the first actuator may be configured for actuating the first pump. Further, the second actuator may be configured for actuating the second pump. Further, the drawing of the first amount of the air from the interior cavity may be based on the actuating of the first pump. Further, the drawing of the at least one second amount of the air from the interior cavity and the blowing of the at least one third amount of the air in the interior cavity may be based on the actuating of the second pump. Further, the at least one valve may be fluidly coupled with the plurality of pumps. Further, the at least one valve may be configured for transitioning between a plurality of valve states based on the reduced pressure. Further, the at least one valve may be configured for allowing at least one of entering and exiting of at least one amount of the air from the interior cavity through the at least one valve based on the transitioning. Further, the allowing of the at least one of the entering and the exiting of the at least one amount of the air from the interior cavity maintains the reduced pressure within a predetermined value range of the reduced pressure. Further, the at least one sensor may be configured for generating at least one sensor data based on a value of a pressure in the body cavity of the individual. Further, the processing device may be communicatively coupled with the at least one sensor. Further, the processing device may be configured for analyzing the at least one sensor data. Further, the analyzing may include comparing the value of the pressure with a predetermined value of the pressure. Further, the processing device may be configured for generating a command based on the analyzing. Further, the processing device may be communicatively coupled with the plurality of actuators. Further, the actuating of the first pump may be based on the command.

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF THE DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the applicants. The applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

FIG. 1 is a schematic diagram of an apparatus for facilitating application of varying pressure to a body of an individual, in accordance with some embodiments.

FIG. 2 is a cross-sectional view of the at least one valve of the apparatus, in accordance with some embodiments.

FIG. 3 is a schematic diagram of the apparatus, in accordance with some embodiments.

FIG. 4 is a schematic diagram of the apparatus, in accordance with some embodiments.

FIG. 5 is a schematic diagram of the apparatus attached to the nasal cavity of the body cavity of the individual, in accordance with some embodiments.

FIG. 6 is a schematic diagram of the apparatus and the nasal cavity, in accordance with some embodiments.

FIG. 7 is a schematic diagram of an apparatus for facilitating application of varying pressure to a body of an individual, in accordance with some embodiments.

FIG. 8 is a cross-sectional view of a nasal mask attached to a nose of an individual for facilitating application of varying pressure to a body of the individual, in accordance with some embodiments.

FIG. 9 is a cross-sectional view of a mask attached to an eye of an individual for facilitating application of varying pressure to a body of the individual, in accordance with some embodiments.

FIG. 10 is a perspective view of a mask for facilitating application of varying pressure to a body of an individual, in accordance with some embodiments.

FIG. 11 is a front view of a plurality of masks attached to a head of an individual for facilitating application of varying pressure to a body of the individual, in accordance with some embodiments.

FIG. 12 is a front view of a mask attached to eyes of an individual for facilitating application of varying pressure to a body of the individual, in accordance with some embodiments.

FIG. 13 is a front view of a plurality of masks attached to ears of an individual for facilitating application of varying pressure to a body of the individual, in accordance with some embodiments.

FIG. 14 is a front view of a mask attached to an eye of an individual for facilitating application of varying pressure to a body of the individual, in accordance with some embodiments.

FIG. 15 is a schematic diagram of an apparatus for facilitating application of varying pressure to a body of an individual, in accordance with some embodiments.

FIG. 16 is an illustration of an online platform consistent with various embodiments of the present disclosure.

FIG. 17 is a block diagram of a computing device for implementing the methods disclosed herein, in accordance with some embodiments.

### DETAIL DESCRIPTIONS OF THE INVENTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim limitation found herein and/or issuing here from that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the claims found herein and/or issuing here from. The present disclosure contains headers. It should be understood that these headers are used as references and are not to be construed as limiting upon the subjected matter disclosed under the header.

The present disclosure includes many aspects and features. Moreover, while many aspects and features relate to, and are described in the context of methods, systems, apparatuses, and devices for facilitating application of varying pressure to a body of an individual, embodiments of the present disclosure are not limited to use only in this context.

In general, the method disclosed herein may be performed by one or more computing devices. For example, in some embodiments, the method may be performed by a server computer in communication with one or more client devices over a communication network such as, for example, the Internet. In some other embodiments, the method may be performed by one or more of at least one server computer, at least one client device, at least one network device, at least one sensor and at least one actuator. Examples of the one or more client devices and/or the server computer may include, a desktop computer, a laptop computer, a tablet computer, a personal digital assistant, a portable electronic device, a wearable computer, a smart phone, an Internet of Things (IoT) device, a smart electrical appliance, a video game console, a rack server, a super-computer, a mainframe computer, mini-computer, micro-computer, a storage server, an application server (e.g. a mail server, a web server, a real-time communication server, an FTP server, a virtual server, a proxy server, a DNS server etc.), a quantum computer, and so on. Further, one or more client devices and/or the server computer may be configured for executing a software application such as, for example, but not limited to, an operating system (e.g. Windows, Mac OS, Unix, Linux, Android, etc.) in order to provide a user interface (e.g. GUI, touch-screen based interface, voice based interface, gesture based interface etc.) for use by the one or more users and/or a network interface for communicating with other devices over a communication network. Accordingly, the server computer may include a processing device configured for performing data processing tasks such as, for example, but not limited to, analyzing, identifying, determining, generating, transforming, calculating, computing, compressing, decompressing, encrypting, decrypting, scrambling, splitting, merging, interpolating, extrapolating, redacting, anonymizing, encoding and decoding. Further, the server computer may include a communication device configured for communicating with one or more external devices. The one or more external devices may include, for example, but are not limited to, a client device, a third party database, public database, a private database and so on. Further, the communication device may be configured for communicating with the one or more external devices over one or more communication channels. Further, the one or more communication channels may include a wireless communication channel and/or a wired communication channel. Accordingly, the communication device may be configured for performing one or more of transmitting and receiving of information in electronic form. Further, the server computer may include a storage device configured for performing data storage and/or data retrieval operations. In general, the storage device may be configured for providing reliable storage of digital information. Accordingly, in some embodiments, the storage device may be based on technologies such as, but not limited to, data compression, data backup, data redundancy, deduplication, error correction, data finger-printing, role based access control, and so on.

Further, one or more steps of the method disclosed herein may be initiated, maintained, controlled and/or terminated based on a control input received from one or more devices operated by one or more users such as, for example, but not limited to, an end user, an admin, a service provider, a service consumer, an agent, a broker and a representative thereof. Further, the user as defined herein may refer to a human, an animal or an artificially intelligent being in any state of existence, unless stated otherwise, elsewhere in the present disclosure. Further, in some embodiments, the one or more users may be required to successfully perform authentication in order for the control input to be effective. In general, a user of the one or more users may perform authentication based on the possession of a secret human readable secret data (e.g. username, password, passphrase, PIN, secret question, secret answer etc.) and/or possession of a machine readable secret data (e.g. encryption key, decryption key, bar codes, etc.) and/or or possession of one or more embodied characteristics unique to the user (e.g. biometric variables such as, but not limited to, fingerprint, palm-print, voice characteristics, behavioral characteristics, facial features, iris pattern, heart rate variability, evoked potentials, brain waves, and so on) and/or possession of a unique device (e.g. a device with a unique physical and/or chemical and/or biological characteristic, a hardware device with a unique serial number, a network device with a unique IP/MAC address, a telephone with a unique phone number, a smartcard with an authentication token stored thereupon, etc.). Accordingly, the one or more steps of the method may include communicating (e.g. transmitting and/or receiving) with one or more sensor devices and/or one or more actuators in order to perform authentication. For example, the one or more steps may include receiving, using the communication device, the secret human readable data from an input device such as, for example, a keyboard, a keypad, a touch-screen, a microphone, a camera and so on. Likewise, the one or more steps may include receiving, using the communication device, the one or more embodied characteristics from one or more biometric sensors.

Further, one or more steps of the method may be automatically initiated, maintained and/or terminated based on one or more predefined conditions. In an instance, the one or more predefined conditions may be based on one or more contextual variables. In general, the one or more contextual variables may represent a condition relevant to the performance of the one or more steps of the method. The one or more contextual variables may include, for example, but are not limited to, location, time, identity of a user associated with a device (e.g. the server computer, a client device etc.) corresponding to the performance of the one or more steps, environmental variables (e.g. temperature, humidity, pressure, wind speed, lighting, sound, etc.) associated with a device corresponding to the performance of the one or more steps, physical state and/or physiological state and/or psychological state of the user, physical state (e.g. motion, direction of motion, orientation, speed, velocity, acceleration, trajectory, etc.) of the device corresponding to the performance of the one or more steps and/or semantic content of data associated with the one or more users. Accordingly, the one or more steps may include communicating with one or more sensors and/or one or more actuators associated with the one or more contextual variables. For example, the one or more sensors may include, but are not limited to, a timing device (e.g. a real-time clock), a location sensor (e.g. a GPS receiver, a GLONASS receiver, an indoor location sensor etc.), a biometric sensor (e.g. a fingerprint sensor), an environmental variable sensor (e.g. temperature sensor, humidity sensor, pressure sensor, etc.) and a device state sensor (e.g. a power sensor, a voltage/current sensor, a switch-state sensor, a usage sensor, etc. associated with the device corresponding to performance of the or more steps).

Further, the one or more steps of the method may be performed one or more number of times. Additionally, the one or more steps may be performed in any order other than as exemplarily disclosed herein, unless explicitly stated otherwise, elsewhere in the present disclosure. Further, two or more steps of the one or more steps may, in some embodiments, be simultaneously performed, at least in part. Further, in some embodiments, there may be one or more time gaps between performance of any two steps of the one or more steps.

Further, in some embodiments, the one or more predefined conditions may be specified by the one or more users. Accordingly, the one or more steps may include receiving, using the communication device, the one or more predefined conditions from one or more and devices operated by the one or more users. Further, the one or more predefined conditions may be stored in the storage device. Alternatively, and/or additionally, in some embodiments, the one or more predefined conditions may be automatically determined, using the processing device, based on historical data corresponding to performance of the one or more steps. For example, the historical data may be collected, using the storage device, from a plurality of instances of performance of the method. Such historical data may include performance actions (e.g. initiating, maintaining, interrupting, terminating, etc.) of the one or more steps and/or the one or more contextual variables associated therewith. Further, machine learning may be performed on the historical data in order to determine the one or more predefined conditions. For instance, machine learning on the historical data may determine a correlation between one or more contextual variables and performance of the one or more steps of the method. Accordingly, the one or more predefined conditions may be generated, using the processing device, based on the correlation.

Further, one or more steps of the method may be performed at one or more spatial locations. For instance, the method may be performed by a plurality of devices interconnected through a communication network. Accordingly, in an example, one or more steps of the method may be performed by a server computer. Similarly, one or more steps of the method may be performed by a client computer. Likewise, one or more steps of the method may be performed by an intermediate entity such as, for example, a proxy server. For instance, one or more steps of the method may be performed in a distributed fashion across the plurality of devices in order to meet one or more objectives. For example, one objective may be to provide load balancing between two or more devices. Another objective may be to restrict a location of one or more of an input data, an output data and any intermediate data therebetween corresponding to one or more steps of the method. For example, in a client-server environment, sensitive data corresponding to a user may not be allowed to be transmitted to the server computer. Accordingly, one or more steps of the method operating on the sensitive data and/or a derivative thereof may be performed at the client device.

### Overview

The present disclosure describes methods, systems, apparatuses, and devices for facilitating application of varying pressure to a body of an individual. The varying pressure is applied to a body cavity of the body and/or a body surface of the body. Further, the present disclosure describes a dual syringe setup for generating a superposed periodic reduced pressure in a nasal cavity is disclosed. It is important to note that the syringe or syringes may be replaced or combined with any method or mechanism useful for withdrawing gases (such as air) or fluids from the desired compartment using a volume displacement mechanism. The dual setup is favourable as the risk of pressure leak is minimised when compared to a single mechanism for drawing, maintaining a reduced pressure as well as then generating an oscillating reduced pressure. The dual setup may better permits for recovery/adjustment of the base line reduced pressure, should a pressure leak occur, and this could be adjusted while maintaining the periodic action more easily due to the separation of activities. While a stepper motor is discussed, any alternative mechanism such as a linear actuator, piezoelectric mechanism, or DC motor and CAM setup may be employed. Alternatively, a diaphragmatic pump could be used to generate the reduced pressure as well as the oscillation. It is also appreciated that combinations of components such as a syringe pump and a diaphragmatic pump could be used. Further, in instances where it would beneficial to have the ability to continuously draw air till a reduced pressure is attained a suction pump could also be employed. Further, the nose of a patient may be connected to a closed system by means of a nasal mask and a head strap, or a nose plug with tubing through the center. It is understood that the other nostril may have the same or similar arrangement or be occluded by another means including a nose plug. Further, the soft palate may be elevated to seal the nasal cavity from the rest of the airway. Further, the elevated soft palate may be achieved by a method such as a forced exhalation method, a mechanical method, an air blow method (blowing air or another gas to elevate soft palate), or an electrical stimulation method. Further, the forced exhalation method may include blowing air through the mouth such that, the force of air pushes the soft palate upwards. The soft palate could also be elevated by other means such as use a utensil or finger to push it upwards. It could then be held in place by application of the systems and methods described above. Additionally, an agent may be administered which would promote seal formation alone or in conjunction with the systems and methods described above. Further, the force may have a minimum threshold or magnitude and be subject to intra-individual variation. Further, the mechanical method may include the pushing on the soft palate by means of an intra-oral appliance with inflating balloons which can act in a posterior as well as upwards direction, pushing the soft palate into a ‘closed’ position creating a seal in the nasal cavity. The balloon array may also act to seal the nasal cavity from the rest of the airway in patients where there is a defect in the soft palate which would prevent seal formation. Further, the air blow method may include elevating the soft palate through tubing housed in an intra-oral appliance. Further, the electrical stimulation method may electrically stimulate the muscles of the soft palate. Further, electrodes may be likely to be housed in an intra-oral appliance. Further, the patient may blow through the mouth this may be augmented by any of the methods mentioned. Further, a pitot-static tube may be reversibly-attached to the nasal mask. Further, the nasal mask may be connected to sensors to detect pressure change as the patient blows. The pitot-static tube may be ‘loose’ i.e. it can be picked up and used when required or indicated. Further, the patient may blow into the pitot-static tube which may activate a linear actuator. Further, the large syringe may draw a fixed volume of air from the nasal cavity. Further, in some embodiments, the components connected to the nasal section may be equipped with a button that may be depressed to activate the system. Further, the system may consist of one or more steps. Further, a first step of the one or more steps may be to instruct forced exhalation for example an audio-visual signal, a tactile signal, or any of these in combination. Further, the desired volume of air to be withdrawn from the nasal cavity may be adjusted at a point of initial fitting of the patient, making the disclosed setup patient-specific. The volume of the nasal cavity may further be determined by means such as acoustic rhinometry. Further, an additional step of the one or more steps may be to increase or decrease the reduced pressure once the seal may be obtained. The pressure may be adjusted to facilitate the desired physiological outcome. Further, the reduced pressure may slowly become more positive until the seal fails. Further, the seal may be maintained (assumed through surface tensions) when a release valve on the apparatus is fully activated or partially activated that is, the whole system may be at atmospheric pressure. The pressure changes can be adjusted by partial activation of the valve (such as analog pressure valves, digital pressure valves, mechanical pressure valves, etc.) either manually or through other means such as digital control. It is also appreciated that a mechanical release valve can be used as a redundancy should the digital valve(s) fail. Further, the mechanical release valve may include one or more valvular components and/or pressure regulating components. Further, the mechanical release valve may include a safety valve. Further, a second stepper motor may actuate a small syringe and further generate a periodic component. Further, a vacuum gauge may sit anywhere between the tubing to the nose, and the small syringe (such as a displacement component/mechanism) and the large syringe (such as a displacement component/mechanism) may display the pressure in the system. Further, the system may include any arrangement of a displacement mechanism as well as a plurality of these components. Further, a flow gauge in-line with a vacuum regulator may indicate airflow and when the vacuum regulating valve setting has been exceeded the action of the syringe, pump, actuator, etc. stops. These components may also act to regulate the pressure in any manner such as reducing the making the pressure in the system more negative. Further, the air may be allowed to flow into the reduced pressure volume as well as in the instance, the reduced pressure has exceeded the pre-set threshold. Further, the flow gauge may indicate whether an adequate seal has been maintained. Further, a vacuum regulator or calibrated vacuum release valve may function to prevent the generation of an excessive high reduced pressure by allowing air to leak in at a pre-set threshold. Further, a digital vacuum or pressure gauge may be used as a feedback mechanism. Further, the digital vacuum may also act to generate the desired pressure without needing to determine other variables such as the volume of the nasal cavity. Further, the digital vacuum gauge may allow for easy setting of operating parameters during initial fitting and any required future adjustments. Further, the digital vacuum gauge may allow the recording of treatment parameters with the inclusion of the requisite electronic components. Further, in some embodiments, a manual pump and/or the addition of a mechanism which may be partially automated or otherwise may be used to generate the requisite pressures with an audio/visual and/or tactile signal to activate the system to generate the periodic component as well as the ‘static’ component which also describes the generation of a non-periodic reduced pressure by action of the displacement mechanism or mechanisms. Further, pressure characteristics may be adjusted based on continuous or intermittent input from a data processor arranged to receive input from a measurement device such as a pressure gauge or a device measuring changes in other bodily structures or functions such as changes in ribcage volume utilizing plethysmography. The components described above are interchangeable, can be numerous, and can lie in any functional sequence to achieve the desired outcome.

The present disclosure describes systems and methods for modifying pressures applied to the cavity or structures of the skull and in particular generating a superposed periodic reduced pressure in a nasal cavity. Further, the aim of the disclosed system may be to create a sealed compartment in the nasal cavity and then to create an oscillatory reduced pressure to stimulate cerebrospinal fluid drainage (CSF) into the nasal lymphatics and/or enhance lymphatic efficiency (presumably through increased force and/or frequency of contraction) as well as by affecting gene expression. By increasing the rate of CSF transfer and its components such as amyloid and other metabolic waste into a nasal compartment, disorders of increased CSF pressure such as hydrocephalus, idiopathic intracranial hypertension as well as Alzheimer's disease, and other neurological disorders may be improved.

By enhancing brain function through clearance of CSF laden metabolites via the cribriform plate, any disease process may potentially be positively influenced through appreciation of the role of the CSF flow and quality on brain processes, directly or indirectly, in disease processes as well during states of wellbeing. The effects may diverse and can include changes in a sympathetic and parasympathetic nervous activity which affect target organs as well as through modification of neuroendocrine activity.

CSF flow across the cribriform plate into the nasal lymphatics is an important outflow route and disruption of this has been shown to increase resting Intra-Cranial Pressure (ICP) in animal models. Furthermore, studies have shown reduced transfer rates into the nasal compartment animal models with increasing age. Studies have also identified a reduction in the number and patency of cribriform foramina with age as well as pathological states such as Alzheimer's disease. Age-related olfactory dysfunction is a recognised phenomenon and is an early sign of the classical neurodegenerative diseases and can reach a prevalence of 100% in Alzheimer's disease patients, 90% in Parkinson's disease, 96% in Frontotemporal Lobe Dementia, and 15% in Vascular dementia. Disruption of CSF flow across the cribriform plate likely predisposes the medial temporal lobe and basal forebrain to pathology when one examines the proximal pathways and structures running to the cribriform ‘outlet’. The proposed intervention attenuates the development of brain pathology by increasing the drainage of CSF across the cribriform plate. Additionally, by facilitating clearance of CSF from the cranial compartment the intracranial pressure can be reduced as well as increase clearance of toxic components present in the CSF. Further, the present disclosure is proposed to treat, improve, retard disease progression and reduce medical complications in Alzheimer's disease as well as other neurological and psychiatric conditions, e.g., Parkinson's disease, frontal-temporal dementia, mild cognitive impairment, idiopathic dementia, vascular dementia, amyotrophic lateral sclerosis, Pick's disease, concussive brain injury, supranuclear palsy, Creutzfeld-Jacob disease and other disorders of protein misfolding, classical and novel disorders involving disturbed CSF hydrodynamics, e.g. hydrocephalus, multiple sclerosis, as well as other neurological and psychiatric disorders including Myalgic Encephalomyelitis, Chronic Fatigue Syndrome, Fibromyalgia, Idiopathic Intracranial Hypertension.

Further, the present disclosure may be used to collect CSF draining across the cribriform plate to diagnose and assess neurological disorders, including Alzheimer's disease as well as other neurological and psychiatric conditions. Olfactory disorders may also be treated directly or as a surrogate indicator of ongoing or pre-disposition to future neuropathology. The individual efficacy of the system can be assessed in combination with delivery of a tracer which can be examined by a PET scan or other imaging modality which would ideally be implemented to determine the rate of transfer between the cranial and nasal compartments pre and post-intervention.

The setup described could be also modified to facilitate fluid exchange, drainage, etc of other structures such as the eye. This would have applications in treating conditions such as glaucoma, which is reported as having a higher incidence in patients with Alzheimer's disease. CSF also travels along the optic nerve such that application of a reduced pressure which may be oscillating may increase drainage to the extracranial compartment. The mechanism of action may vary and may even act in a variety of ways such as increasing clearance of aqueous humour into the Schlemm canal. It may also facilitate ‘unblocking’ of the Schlemm canal. The mechanism may also act by affecting gene expression via mechanotransduction. The Schlemm canal has been described as having a lymphatic phenotype which would make it amenable to physiological entrainment. The pressure can be applied to both eyes using a modified pair of goggles, each eye independently or to one eye alone—employing an appropriate form of enclosure. The pressure when applied to any targeted structure may be constant, oscillating, oscillating upon a constant pressure, decaying, gradually increasing, or of other characteristics. When targeting each eye independently the appropriate waveform and pressure can be applied to better optimize treatment.

The ear could also be easily targeted and may positively influence CSF drainage along the vestibular nerve. It has been shown that external pressure changes influence inner ear pressures most commonly appreciated by air travelers as ‘blocked ear’ due to closure of the Eustachian tube and failure of pressure equalisation. Selectively and carefully manipulating inner ear pressure is proposed to positively CSF transfer from the perineural compartment to the perilymph spaces of the inner ear and into the lymphatic system of the middle ear. The facial nerve also drains into the perilymph of the inner ear. The lymphatic drainage of the inner ear is also expected to be affected by manipulation of the applied pressure and may require a differing pressure amplitude, waveform, etc. to optimally facilitate drainage. This may be applied alone or intermittently with the treatment targeted towards perineural transfer of CSF. The connection to the ear may also be in a number of forms such as an ear plug with a tube sitting flush or extending as far as desired. These components can also be custom moulded and modular as with all the other designs discussed e.g. nasal system. The system could also act to assist in evacuation of a blocked ear canal. When the system is applied to different areas the treatment can be synchronized as well as adjusted based on continuous or intermittent input from a data processor.

Further, the disclosed system may be simplified for assisting the evacuation of the nasal sinus either through a continuous or oscillating reduced pressure once the nasal compartment has been sealed. Further, the disclosed setup may have a chamber for collection. Once the seal has been maintained a therapeutic gas such as hydrogen gas may be instilled into the nasal cavity at a pressure less than that required to break the soft palate seal. Further, an in-line pressure gauge may indicate a fall in pressure and the system may respond accordingly. Further, the hydrogen gas may be instilled to maintain the soft palate seal (Stiction seal) by first generating a reduced pressure which becomes more positive as the gas is instilled. Tubing inserting into the nose can be of differing lengths and moulded/shaped to fit further into the nose. If the cavity is under a reduced pressure instillation of gas will experience reduced resistance to diffusion. Should a volume of air be displaced such as withdrawal of the syringe plunger generating a more reduced pressure the gas would more likely be evacuated through the vacuum outlet. The instillation tube could be inserted further such that the distribution to the target structures such as the olfactory nerves would have greater priority under most circumstances. The ‘inflow’ and ‘outflow’ tubes can be in each nostril or share the same nostril. Further, in some embodiments, the hydrogen gas may be infused on a more positive pressure. Hydrogen gas has been shown to have a number of beneficial effects acting as a small, highly permeable antioxidant as well as by affecting signalling pathways, rescue of injured, ischaemic tissue as well as tissue regeneration including nerves. It is proposed that instilling hydrogen gas at the desired concentration into a sealed compartment is more effective at increasing gas concentration throughout the whole of the nasal cavity within the structures such as the nasal lymphatics, olfactory nerve, cribriform bone as well as gaseous transfer into the cranial compartment. The efficacy over inhalation via a nasal cannula can be appreciated by the basic understanding of gas kinetics in open versus closed compartments.

Further, the soft palate must be elevated to seal the nasal cavity from the rest of the airway. Further, the elevated soft palate may be achieved by a method such as an electrical stimulation method in the form of an implantable, removable system, system with removable parts, or a combination thereof. Further, the electrical stimulation method may electrically stimulate the muscles of the soft palate. Further, the electrical stimulation method may have effects on snoring and obstructive sleep apnea (OSA). Further, OSA is a disorder characterised by repetitive complete or partial collapse of the upper airways (tongue and soft palate and other oro-pharyngeal structures) during sleep in spite of respiratory effort. and has been associated with social and physical problems and is predicted to rise. In 2015, 5.9 million patients were diagnosed with OSA in the U.S. with an estimated 23.5 million undiagnosed. Many treatments of the OSA have been proposed with various levels of success but most are obtrusive, cumbersome, or invasive. It is also important to appreciate the role of sleep disordered breathing in neurodegenerative disorders such as Alzheimer's. Any benefits on soft palate function in sleep disordered breathing are considered secondary to the desired closure of the soft palate during application of the described methods within. Strengthening the soft palate by electrical stimulation may permit temporary or permanent cessation of the electrical device and allow use of other methods of soft palate closure such as the exhalation method. Seven patients with histories of snoring and the OSA participated in a pilot study to evaluate the effects of electrical stimulation on the soft palate. Each patient slept with a palatal appliance that delivered a weak electrical stimulus to the soft palate on activation. A 3 milliampere stimulus in the range of 9 to 10 volts was found to be effective in terminating snoring without causing patient arousal. The effects of the stimulus on the OSA were variable. Further, the electrical stimulation of the soft palate may be effective as a treatment for snoring and OSA.

According to some embodiments, a dual syringe setup for generating a superposed periodic reduced pressure in a nasal cavity of a patient is disclosed.

According to some embodiments, a single syringe setup for generating a superposed periodic reduced pressure in a nasal cavity of a patient is disclosed.

Further, the disclosed setup (the dual syringe setup, the single syringe setup, or variations using other mechanisms) may be used with other devices including respiratory devices such as an Impedance Threshold Device (ITD) or Intrathoracic Pressure Regulator (IPR). By mildly increasing resistance to breathing it increases negative intrathoracic pressure, which increases venous return to the heart. By increasing the negative intrathoracic pressure, increased lymph may enter the circulation affecting lymph flow upstream facilitating clearance of increased lymph in cranial lymphatics as a result of the systems and methods described.

Further, the disclosed setup may be used in states of reduced physiological cerebrospinal fluid drainage such as in zero gravity. It is recognized prolonged exposure to zero gravity disturbs CSF hydrodynamics and contributes to Visual Impairment Intracranial Pressure syndrome (VIIP)— this has recently been re-defined by NASA as Spaceflight-Associated Neuro-ocular Syndrome (SANS). One of the proposed mechanisms is mild but chronic increase in CSF pressure leading to a mismatch between ICP and intra-ocular pressure driving pathology. The system could also be modified to generate clearance rates which may approximate the range of ICP's seen on earth during positional changes and activities. The waveform of the generated pressure may be regular, regularly irregular, irregularly irregular, etc. permitting a full range of therapeutic applications. It can be further coupled to temporal and activity states. Further, the disclosed setup may be used to enhance physiological clearance of CSF after prolonged wakefulness or disturbed sleep which has impaired the activity of the glymphatic system and possibly impaired CSF flow across the cribriform plate into the lymphatics. The glymphatic system and cranial lymphatic system are likely to have dependent roles. Furthermore, the disclosed setup may be employed where there is no overt pathology, but where its use may optimize physiology in the instance of the aging adult.

Further, the present disclosure describes a dual syringe setup for generating a superposed periodic reduced pressure in a nasal cavity of a patient. The nose of a patient may be connected to the dual syringe setup (a closed system) by means of a nasal mask and a head strap, or a nose plug with tubing through the center. Further, the soft palate may be elevated to seal the nasal cavity from the rest of the airway. Further, the elevated soft palate may be achieved by a method such as a forced exhalation method, a mechanical method, an air blow method, or an electrical stimulation method. Further, the forced exhalation method may include blowing air through the mouth such that, the force of air pushes the soft palate upwards. Further, the force may have a minimum threshold or magnitude and be subject to intra-individual variation. Further, the mechanical method may include the pushing on the soft palate by means of an intra-oral appliance with inflating balloons which can act in a posterior as well as upwards direction. Further, the air blow method may include elevating the soft palate through tubing housed in an intra-oral appliance. Further, the electrical stimulation method may electrically stimulate the muscles of the soft palate. Further, electrodes may be likely to be housed in an intra-oral appliance. Further, the patient may blow through the mouth, thereby elevating the soft palate and creating a seal. Further, the patient may blow into a pitot-static tube (of the dual syringe setup) attached to the nasal mask connected to sensors. As the patient blows, the sensors may detect the pressure change. This may activate a linear actuator (of the dual syringe setup) for a large syringe that draws a fixed volume of air from the nasal cavity. In some embodiments, once a nasal section is placed, a button (of the dual syringe setup) may be depressed to activate the dual syringe setup, wherein the first step may include instructing forced exhalation by an audio-visual signal. Thereafter, the desired volume of the nasal cavity may be adjusted at a point of initial fitting making the device (the dual syringe setup) patient-specific. Further, an additional step may include increasing or decreasing the reduced pressure (possibly out of a need of a more physiological pressure range) once the seal is obtained, that is, slowly becomes more positive till the seal fails. Further, the seal may be maintained (assumed through surface tensions) when a release valve on a handheld vacuum pump is fully activated that is, the whole system (the dual syringe setup) may be at atmospheric pressure or a relatively more positive pressure. Further, a second stepper motor (of the dual syringe setup) may actuate a small syringe and further generate a periodic component. Further, a vacuum gauge may sit between the tubing to the nose, and the small syringe and the large syringe may display the pressure in the system (the dual syringe setup). Further, a flow gauge in line with the vacuum regulator may indicate air flow through the vacuum regulator when the predetermined vacuum regulating valve setting is exceeded and air is allowed into the reduced pressure volume. This may indicate whether an adequate seal has been maintained. Further, an in-line vacuum regulator may function to prevent the generation of an excessive high reduced pressure by allowing air to leak in at a pre-set threshold. Further, a digital vacuum gauge may be used as a feedback mechanism. Further, the digital vacuum gauge may allow for easy setting of operating parameters during initial fitting and any required future adjustments. Further, the digital vacuum gauge may allow the recording of treatment parameters with the inclusion of the requisite electronic components. Further, in some embodiments, a manual pump may be used to generate the requisite pressures with an audio/visual and/or tactile signal to activate the system to generate the periodic component. Further, pressure characteristics may be adjusted based on continuous or intermittent input from a data processor arranged to receive input from a measurement device such as a pressure gauge or a device measuring changes in other bodily structures or functions such as changes in ribcage volume utilizing plethysmography. In some embodiments, the dual syringe setup may include a processing device, a communication device, and a storage device. The processing device may be configured to receive and process data received from the sensors. Further, the processing device may be configured to execute software instructions.

Further, the present disclosure describes a single syringe setup for generating a superposed periodic reduced pressure in a nasal cavity. Further, the nose of a patient may be connected to the single syringe setup (a closed system) by means of a nasal mask and a head strap, or a nose plug with tubing through the center. Further, the soft palate may be elevated to seal the nasal cavity from the rest of the airway. Further, the elevated soft palate may be achieved by a method such as a forced exhalation method, a mechanical method, an air blow method, or an electrical stimulation method. Further, the forced exhalation method may include blowing air through the mouth such that, the force of air pushes the soft palate upwards. Further, the force may have a minimum threshold or magnitude to have intra-individual variation. Further, the mechanical method may include the pushing on the soft palate by means of an intra-oral appliance with posterior inflating balloons. Further, the air blow method may include elevating the soft palate through tubing housed in an intra-oral appliance. Further, the electrical stimulation method may electrically stimulate the muscles of the soft palate. Further, electrodes may be likely to be housed in an intra-oral appliance. Further, the patient may blow through the mouth, thereby elevating the soft palate and creating a seal. The steps to instruct the aforementioned actions may be coupled with the activity of the system (the single syringe setup). Further, a driving mechanism may activate a mini-syringe drawing air out generating a reduced pressure maintaining a seal. Further, an interchangeable release valve that may sit anywhere in the system depending on the pressure required to maintain the seal and peak amplitude of oscillatory reduced pressure, may be decided for a particular patient. The valve or disposable device may be changed during the initial fitting. A digital pressure release valve may also be used. Further, a vacuum gauge may sit between the tubing to the nose and the syringe may display the pressure in the system (the single syringe setup). Further, a flow gauge may indicate whether an adequate seal has been maintained. Further, in some embodiments, a digital vacuum gauge may be used as a feedback mechanism which may allow for easy setting of operating parameters during initial fitting and any required future adjustments as well as allowing the recording of treatment parameters with the inclusion of requisite electronic components. Further, in some embodiments, a manual pump may be used to generate the requisite pressures with an audio/visual and/or tactile signal to activate the system to generate the periodic component. Further, pressure characteristics may be adjusted based on a continuous or an intermittent input from a data processor arranged to receive input from a measurement device, such as a pressure gauge or a device measuring changes in other bodily structures or functions, such as changes in ribcage volume utilizing plethysmography. In some embodiments, the single syringe setup may include a processing device, a communication device, and a storage device. The processing device may be configured to receive and process data received from the sensors. Further, the processing device may be configured to execute software instructions.

Further, the present disclosure describes an appliance in place over the hard palate which extends onto a soft palate. A device may be retained by wire clasps or other securing mechanisms. In addition, two electrodes, one on either side of the middle of the soft palate are present.

Further, the present disclosure describes a dual syringe setup for generating a superposed periodic reduced pressure in a nasal cavity. Further, the dual syringe setup may include a digital vacuum gauge, a vacuum regulator, a flow gauge, an in-line vacuum regulator, and a vacuum gauge.

Further, the present disclosure describes a single syringe setup for generating a superposed periodic reduced pressure in a nasal cavity. Further, the single syringe setup may include an oscillating mini-syringe, a vacuum gauge, a flow gauge, and a digital vacuum gauge.

Further, the present disclosure describes a nasal mask and an oral mask. Further, the nasal mask and the oral mask can be separate or sub-structures within one mask. Further, the nasal mask and the oral mask may be contained within the same cavity. Further, in some embodiments, a pitot-static tube may be attached to the nasal mask.

Further, the present disclosure describes eye, nasal, and ear masks/pieces attached over a user. Further, the ear pieces may include tubing contained within a head band. Further, the pressure required to be generated for each ear may be the same. Further, the nasal section may be attached to the eye piece. Further, the eye, nasal, and ear masks/pieces may be used in synchronicity as well as be adjusted by continuous or intermittent input from the data processor.

Further, the present disclosure describes goggles for both eyes of a user with independent chambers and tubing. Further, the googles may include independent eye compartments with tubing attached to each compartment.

Further, the present disclosure describes earpieces for both ears with independent tubing attached over a user.

Further, the present disclosure describes an eye mask over one eye of a user. Further, a strap may be attached to the eye piece.

Further, the present disclosure relates generally to medical equipment. More specifically, the present invention is systems and methods for modifying pressures applied to cavities or structures of the skull and in particular generating a superposed periodic reduced pressure in a nasal cavity.

Further, the present disclosure describes systems and methods for modifying pressures applied to cavities or structures of the skull and in particular generating a superposed periodic reduced pressure in a nasal cavity.

Further, the present disclosure describes a method for modifying pressures applied to cavities or structures of the skull and in particular generating a superposed periodic reduced pressure in a nasal cavity. Further, the method may include sealing nose connector to the nose. Blow out through the mouth. This will seal the nasal cavity at the back of the nasal cavity by lifting the soft palate. If a pitot static tube or other air flow sensor is installed, they will detect the patient blowing and start the device. If not installed then the patient will manually start the device. The first stage is volume evacuation of the sealed nasal cavity. This may be performed by a single or multiple “large syringes” or “small syringes”. Further, these syringes are a form of a volume displacement device for volume evacuation. Further, the volume that is to be evacuated by the “large syringes” is in excess of the nasal cavity. If uncontrolled this may produce an undesirably reduced pressure in the nasal cavity. To manage this a preset calibrated vacuum release valve is used. Further, the present calibrated vacuum release valve will allow air from outside of the system to “leak” into the system until the preset calibrated vacuum release valve is at its preset level. The control of the reduced pressure is a critical condition. If a volume of hydrogen gas is to be instilled into the nasal cavity, to maintain the reduced pressure in the nasal cavity, the syringes will need to retract further to enlarge the system volume to maintain the system's reduced pressure. There is no net flow of gases in or out of the system at this stage. The Hydrogen gas will diffuse through the air in the system. When the soft palate lifts and seals the nasal cavity, the nasal cavity and device become a sealed system unless the pressure is too low and ambient air is allowed to leak into the system. Therefore, there is no net flow of gas in or out of the sealed system unless the preset calibrated vacuum release valve is activated. The next phase is to oscillate the reduced pressure within the system. The pressure will be very slightly increased then returned to its original setting for oscillating the pressure. If the pressure is reduced then the preset calibrated vacuum release valve will activate. This change of pressure can be actioned by a secondary smaller syringe or even the primary larger syringe (or any combination of small, large, primary, and secondary syringes) depending on what resolution of pressure change is required. The frequency of reduced pressure changes required will be determined by the combination of the size of syringes and volume displacements required and the frequency of oscillation of the stepper motor. This can be modified by having multiple syringes activated in parallel. Further, the preset calibrated vacuum release valve can be connected to any part of the closed pneumatic. It does not have to be in line with the syringes.

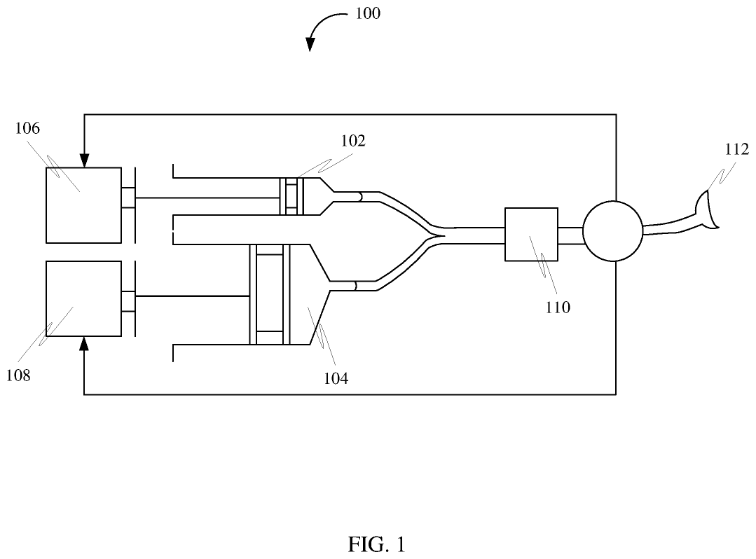

FIG. 1 is a schematic diagram of an apparatus 100 for facilitating application of varying pressure to a body of an individual, in accordance with some embodiments. Further, the apparatus 100 may include a plurality of pumps 102-104, a plurality of actuators 106-108, and at least one valve 110.

Further, the plurality of pumps 102-104 may be configured to be fluidly coupled with the body of the individual using a coupling assembly 112. Further, the coupling assembly 112 may be configured to be attached to the body. Further, the coupling assembly 112 may include an interior cavity. Further, the interior cavity may be coupled with a body cavity of the individual. Further, the plurality of pumps 102-104 may include a plurality of syringe pumps, a plurality of diaphragmatic pumps, etc. Further, the coupling assembly 112 may include a mask. Further, the plurality of pumps 102-104 may include a first pump and a second pump. Further, the first pump may be configured for drawing a first amount of air from the interior cavity for generating a reduced pressure of a first value in the body cavity. Further, the reduced pressure may be less than an ambient pressure. Further, the ambient pressure may be the atmospheric pressure. Further, the second pump may be configured for drawing at least one second amount of the air from the interior cavity and blowing at least one third amount of the air in the interior cavity for varying the reduced pressure in the body cavity between at least one second value and at least one third value from the first value. Further, the body cavity may be associated with at least one part of a body of the individual. Further, the at least one part of the body may include eyes, ears, a nose, a mouth, etc.

Further, the plurality of actuators 106-108 may include a first actuator and a second actuator. Further, the first actuator may be operationally coupled with the first pump and the second actuator may be operationally coupled with the second pump. Further, the first actuator may be configured for actuating the first pump. Further, the second actuator may be configured for actuating the second pump. Further, the drawing of the first amount of the air from the interior cavity may be based on the actuating of the first pump. Further, the drawing of the at least one second amount of the air from the interior cavity and the blowing of the at least one third amount of the air in the interior cavity may be based on the actuating of the second pump.

Further, the at least one valve 110 may be fluidly coupled with the plurality of pumps 102-104. Further, the at least one valve 110 may include a vacuum release valve. Further, the at least one valve 110 may be configured for transitioning between a plurality of valve states based on the reduced pressure. Further, the at least one valve 110 may be configured for allowing at least one of entering and exiting of at least one amount of the air from the interior cavity through the at least one valve 110 based on the transitioning. Further, the allowing of the at least one of the entering and the exiting of the at least one amount of the air from the interior cavity maintains the reduced pressure within a predetermined value range of the reduced pressure.

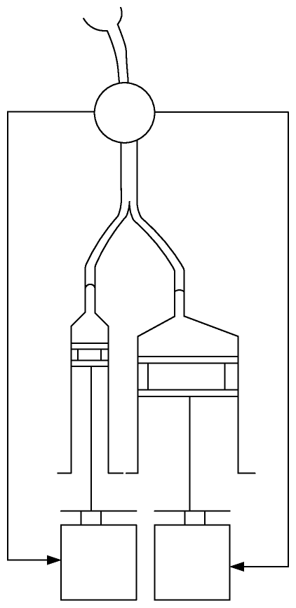

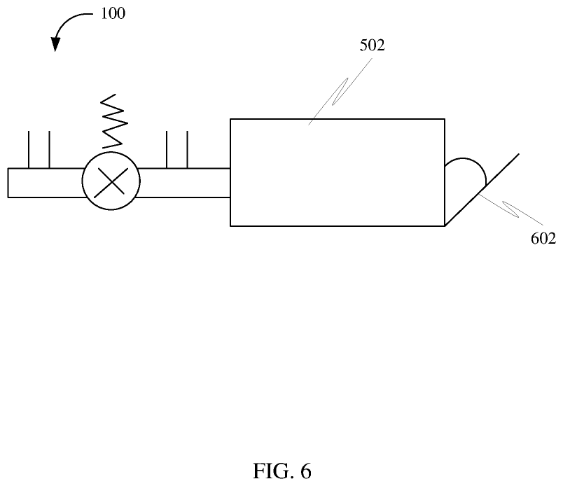

In further embodiments, an intra oral appliance may be configured for pushing a soft palate 602, as shown in FIG. 6, present in a nasal cavity 502, as shown in FIG. 5 and FIG. 6, of the body cavity for sealing the nasal cavity 502 from at least one airway associated with the nasal cavity 502. Further, the drawing of the first amount of the air from the interior cavity may be based on the sealing. Further, the drawing of the at least one second amount of the air from the interior cavity and the blowing of the at least one third amount of the air in the interior cavity may be based on the sealing. Further, in an embodiment, the intra oral appliance may include at least one inflatable balloon. Further, the intra oral appliance may be configured for inflating the at least one inflatable balloon. Further, the pushing of the soft palate 602 may be based on inflating of the at least one inflatable balloon. Further, in an embodiment, the intra oral appliance may include at least one tube. Further, the intra oral appliance may be configured for blowing a gas through the at least one tube towards the soft palate 602. Further, the pushing of the soft palate 602 may be based on the blowing of the gas through the at least one tube. Further, in an embodiment, the intra oral appliance may include at least one electrode. Further, the at least one intra oral appliance may be configured for electrically stimulating the soft palate 602 using the at least one electrode. Further, the pushing of the soft palate 602 may be based on the electrically stimulating of the soft palate 602 using the at least one electrode.

Further, in some embodiments, the varying of the reduced pressure between at least one second value and at least one third value from the first value may be associated with a frequency. Further, in an embodiment, the frequency varies between a plurality of frequency values of the frequency.

Further, in some embodiments, the varying of the reduced pressure between at least one second value and at least one third value from the first value may be associated with a rate. Further, in an embodiment, the rate varies between a plurality of rate values of the rate.

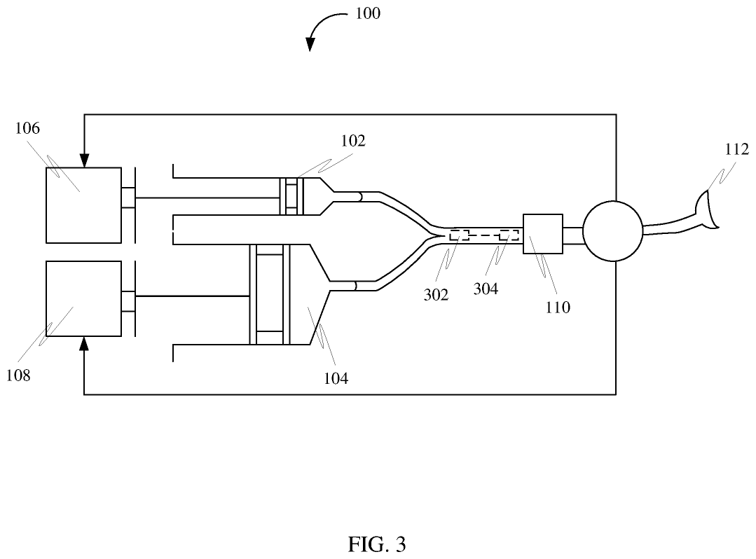

In further embodiments, the apparatus 100 may include a measurement device 302 and a processing device 304, as shown in FIG. 3. Further, the measurement device 302 may be configured for generating measurement data based on at least one bodily function of the individual. Further, the processing device 304 may be communicatively coupled with the measurement device 302. Further, the processing device 304 may be configured for analyzing the measurement data. Further, the processing device 304 may be configured for determining the first value of the reduced pressure based on the analyzing. Further, the processing device 304 may be configured for determining the at least one second value and the at least one third value of the reduced pressure based on the analyzing. Further, the processing device 304 may be communicatively coupled with the plurality of actuators 106-108. Further, the actuating of the first pump may be based on the determining of the first value. Further, the actuating of the second pump may be based on the determining of the at least one second value and the at least one third value. Further, in an embodiment, the measurement device 302 may be communicatively coupled with a communication device. Further, the communication device may be configured for transmitting the measurement data to at least one user device. Further, the at least one user device may be configured for presenting the measurement data.

In further embodiments, the apparatus 100 may include a gas instilling device and a gas instilling device actuator. Further, the gas instilling device may be fluidly coupled with the interior cavity of the coupling assembly 112. Further, the gas instilling device may be configured for instilling at least one fourth amount of a therapeutic gas in the interior cavity. Further, the therapeutic gas may include hydrogen gas. Further, the gas instilling device actuator may be operationally coupled with the gas instilling device. Further, the gas instilling device actuator may be configured for actuating the gas instilling device. Further, the instilling of the at least one fourth amount of the therapeutic gas may be based on the actuating of the gas instilling device. Further, in an embodiment, the gas instilling device may be operationally coupled with the first pump. Further, the first pump may be configured for drawing the at least one fourth amount of the air from the interior cavity based on the instilling of the of the at least one fourth amount of the therapeutic gas. Further, the drawing of the at least one fourth amount of the air from the interior cavity maintains the reduced pressure of the first value.

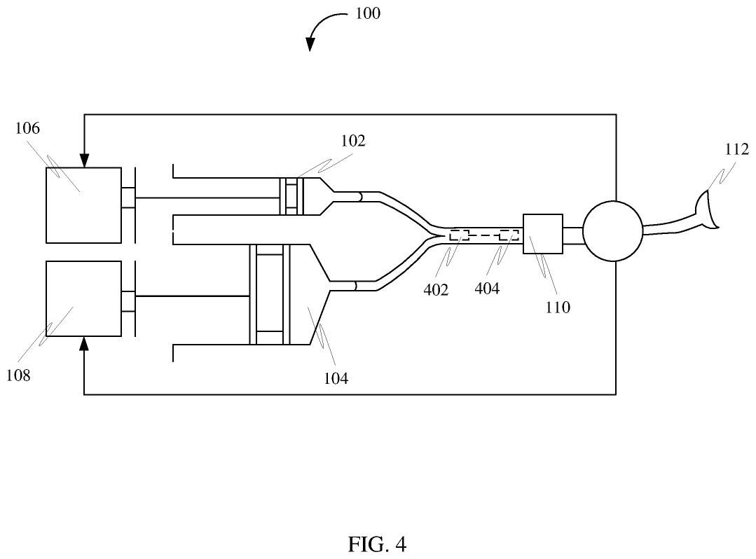

In further embodiments, the apparatus 100 may include at least one sensor 402 and a processing device 404, as shown in FIG. 4. Further, the at least one sensor 404 may be configured for generating at least one sensor data based on a value of a pressure in the body cavity of the individual. Further, the processing device 404 may be communicatively coupled with the at least one sensor 402. Further, the processing device 404 may be configured for analyzing the at least one sensor data. Further, the analyzing may include comparing the value of the pressure with a predetermined value of the pressure. Further, the processing device 404 may be configured for generating a command based on the analyzing. Further, the processing device 404 may be communicatively coupled with the plurality of actuators 106-108. Further, the actuating of the first pump may be based on the command. Further, in an embodiment, the at least one sensor 402 may be communicatively coupled with a communication device. Further, the communication device may be configured for transmitting the at least one sensor data to at least one user device. Further, the at least one user device may be configured for presenting the at least one sensor data.

Further, in some embodiments, the interior cavity may be coupled to a body surface of the individual. Further, the drawing of the first amount of the air from the interior cavity generates the reduced pressure on the body surface. Further, the drawing of the at least one second amount of the air from the interior cavity and the blowing of the at least one third amount of the air in the interior cavity varies the reduced pressure on the body surface between at least one second value and at least one third value.

Further, in some embodiments, a pump of the plurality of pumps 102-104 may include a single syringe pump. Further, the single syringe pump may be configured for drawing the first amount of the air from the interior cavity for generating the reduced pressure of the first value in the body cavity. Further, the single syringe pump may be configured for drawing the at least one second amount of the air from the interior cavity and blowing the at least one third amount of the air in the interior cavity for varying the reduced pressure in the body cavity between the at least one second value and the at least one third value from the first value.

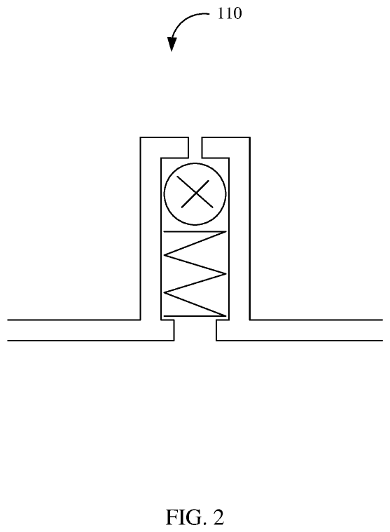

FIG. 2 is a cross-sectional view of the at least one valve 110 of the apparatus 100, in accordance with some embodiments.

FIG. 3 is a schematic diagram of the apparatus 100, in accordance with some embodiments.

FIG. 4 is a schematic diagram of the apparatus 100, in accordance with some embodiments.

FIG. 5 is a schematic diagram of the apparatus 100 attached to the nasal cavity 502 of the body cavity of the individual, in accordance with some embodiments.

FIG. 6 is a schematic diagram of the apparatus 100 and the nasal cavity 502, in accordance with some embodiments.

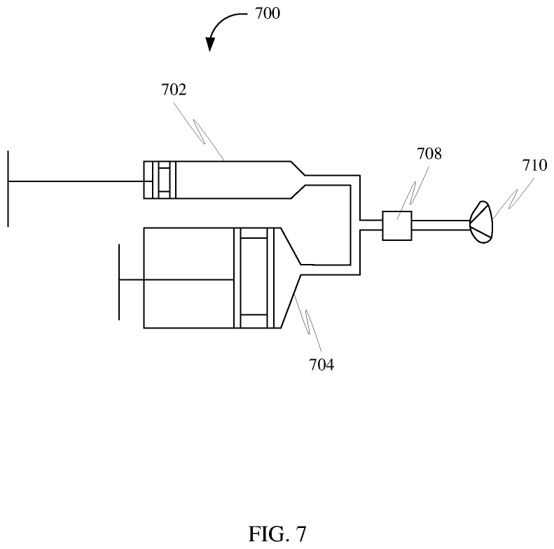

FIG. 7 is a schematic diagram of an apparatus 700 for facilitating application of varying pressure to a body of an individual, in accordance with some embodiments. Further, the apparatus 700 may include a plurality of pumps 702-704, at least one valve 708, and a mask 710.

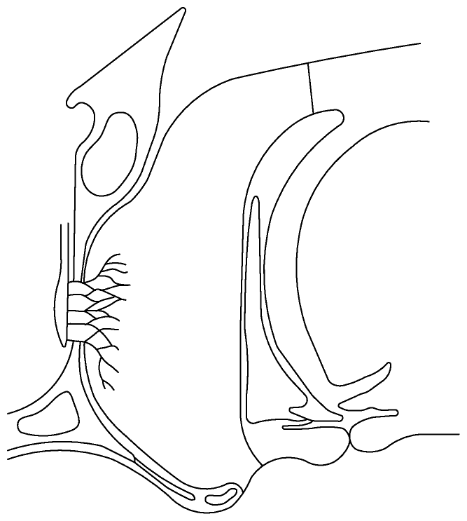

FIG. 8 is a cross-sectional view of a nasal mask 802 attached to a nose of an individual for facilitating application of varying pressure to a body of the individual, in accordance with some embodiments.

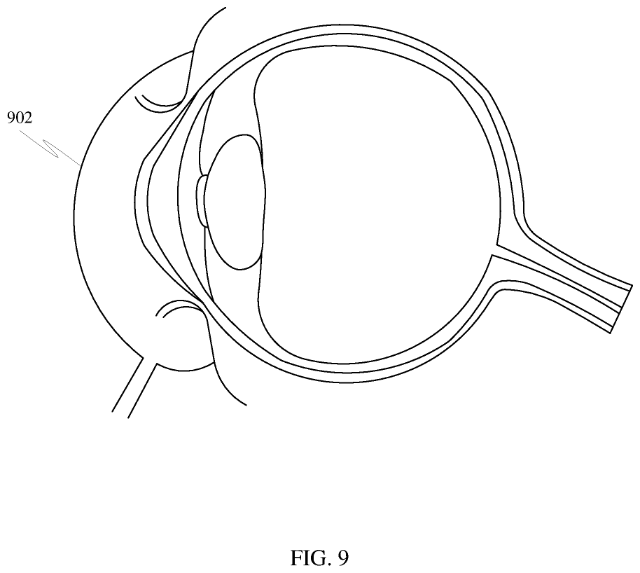

FIG. 9 is a cross-sectional view of a mask 902 attached to an eye of an individual for facilitating application of varying pressure to a body of the individual, in accordance with some embodiments.

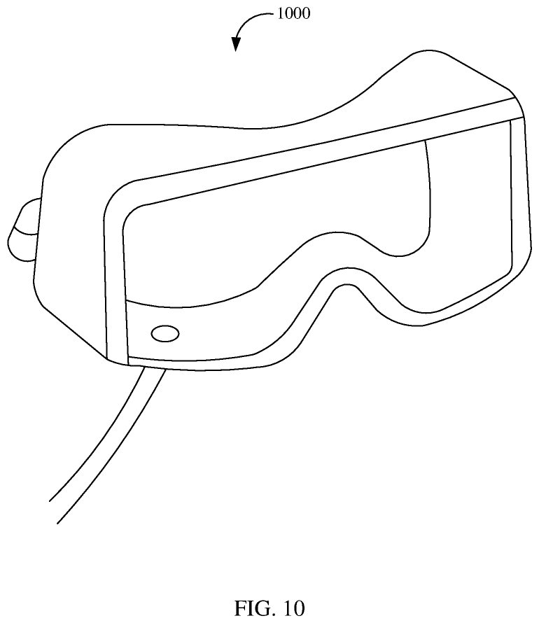

FIG. 10 is a perspective view of a mask 1000 for facilitating application of varying pressure to a body of an individual, in accordance with some embodiments.

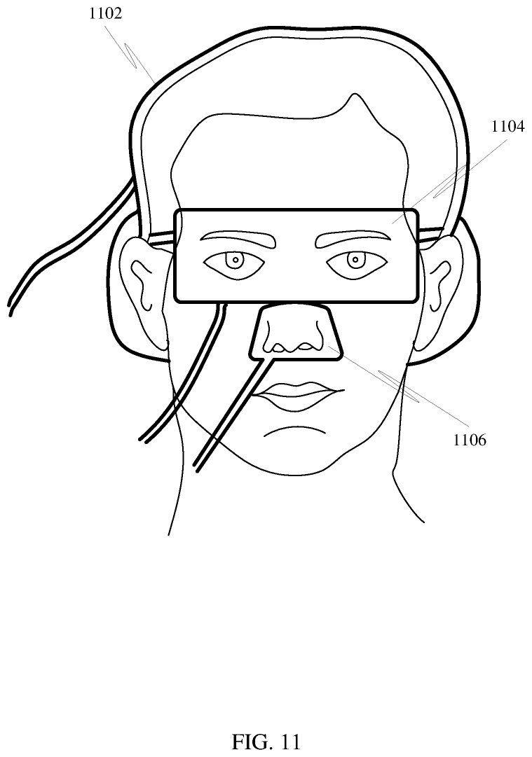

FIG. 11 is a front view of a plurality of masks 1102-1106 attached to a head of an individual for facilitating application of varying pressure to a body of the individual, in accordance with some embodiments. Further, the varying pressure may be applied to a body cavity of the body and a body surface of the body.

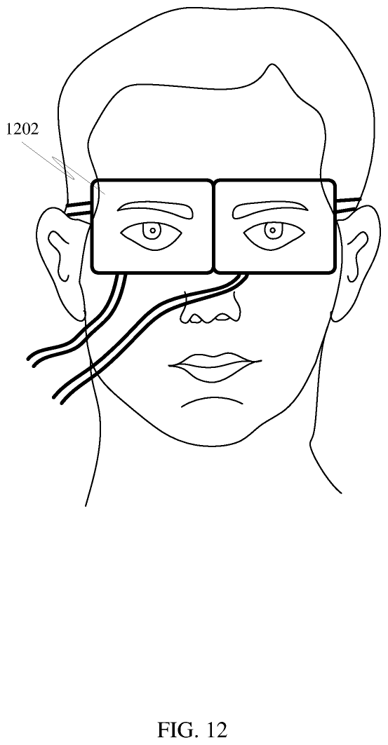

FIG. 12 is a front view of a mask 1202 attached to eyes of an individual for facilitating application of varying pressure to a body of the individual, in accordance with some embodiments.

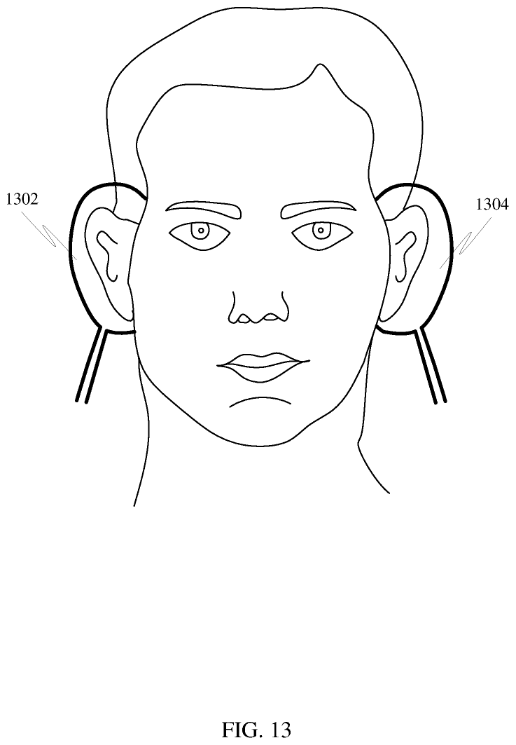

FIG. 13 is a front view of a plurality of masks 1302-1304 attached to ears of an individual for facilitating application of varying pressure to a body of the individual, in accordance with some embodiments.

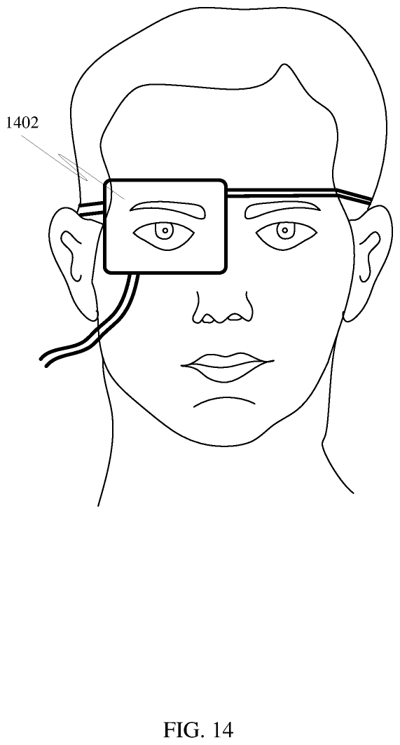

FIG. 14 is a front view of a mask 1402 attached to an eye of an individual for facilitating application of varying pressure to a body of the individual, in accordance with some embodiments.

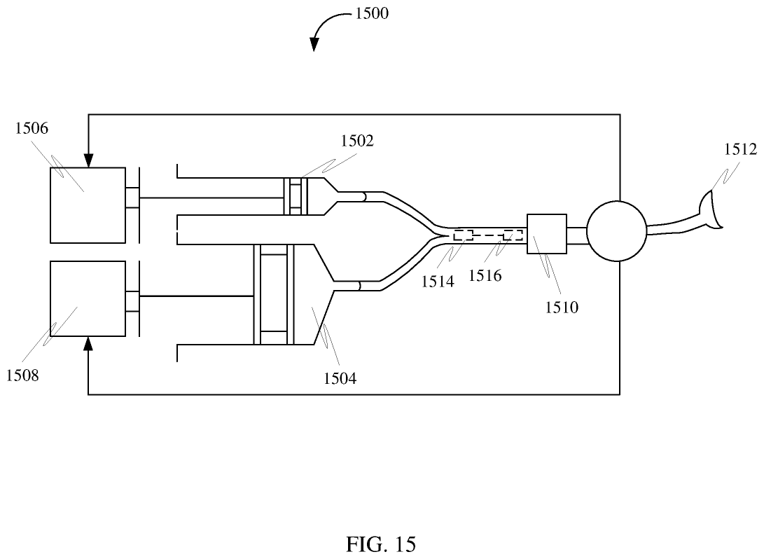

FIG. 15 is a schematic diagram of an apparatus 1500 for facilitating application of varying pressure to a body of an individual, in accordance with some embodiments. Further, the apparatus 1500 may include a plurality of pumps 1502-1504, a plurality of actuators 1506-1508, at least one valve 1510, at least one sensor 1514, and a processing device 1516.

Further, the plurality of pumps 1502-1504 may be configured to be fluidly coupled with the body of the individual using a coupling assembly 1512. Further, the coupling assembly 1512 may be configured to be attached to the body. Further, the coupling assembly 1512 may include an interior cavity. Further, the interior cavity may be coupled with a body cavity of the individual. Further, the plurality of pumps 1502-1504 may include a first pump and a second pump. Further, the first pump may be configured for drawing a first amount of air from the interior cavity for generating a reduced pressure of a first value in the body cavity. Further, the second pump may be configured for drawing at least one second amount of the air from the interior cavity and blowing at least one third amount of the air in the interior cavity for varying the reduced pressure in the body cavity between at least one second value and at least one third value from the first value.

Further, the plurality of actuators 1506-1508 may include a first actuator and a second actuator. Further, the first actuator may be operationally coupled with the first pump and the second actuator may be operationally coupled with the second pump. Further, the first actuator may be configured for actuating the first pump. Further, the second actuator may be configured for actuating the second pump. Further, the drawing of the first amount of the air from the interior cavity may be based on the actuating of the first pump. Further, the drawing of the at least one second amount of the air from the interior cavity and the blowing of the at least one third amount of the air in the interior cavity may be based on the actuating of the second pump.

Further, the at least one valve 1510 may be fluidly coupled with the plurality of pumps 1502-1504. Further, the at least one valve 1510 may be configured for transitioning between a plurality of valve states based on the reduced pressure. Further, the at least one valve 1510 may be configured for allowing at least one of entering and exiting of at least one amount of the air from the interior cavity through the at least one valve 1510 based on the transitioning. Further, the allowing of the at least one of the entering and the exiting of the at least one amount of the air from the interior cavity maintains the reduced pressure within a predetermined value range of the reduced pressure.

Further, the at least one sensor 1514 may be configured for generating at least one sensor data based on a value of a pressure in the body cavity of the individual.

Further, the processing device 1516 may be communicatively coupled with the at least one sensor 1514. Further, the processing device 1516 may be configured for analyzing the at least one sensor data. Further, the analyzing may include comparing the value of the pressure with a predetermined value of the pressure. Further, the processing device 1516 may be configured for generating a command based on the analyzing. Further, the processing device 1516 may be communicatively coupled with the plurality of actuators 1506-1508. Further, the actuating of the first pump may be based on the command.

In further embodiments, an intra oral appliance may be configured for pushing a soft palate present in a nasal cavity of the body cavity for sealing the nasal cavity from at least one airway associated with the nasal cavity. Further, the drawing of the first amount of the air from the interior cavity may be based on the sealing. Further, the drawing of the at least one second amount of the air from the interior cavity and the blowing of the at least one third amount of the air in the nasal cavity may be based on the sealing.

In further embodiments, the apparatus 1500 may include a measurement device and a processing device. Further, the measurement device may be configured for generating measurement data based on at least one bodily function of the individual. Further, the processing device may be communicatively coupled with the measurement device. Further, the processing device may be configured for analyzing the measurement data. Further, the processing device may be configured for determining the first value of the reduced pressure based on the analyzing. Further, the processing device may be configured for determining the at least one second value and the at least one third value of the reduced pressure based on the analyzing. Further, the processing device may be communicatively coupled with the plurality of actuators 1506-1508. Further, the actuating of the first pump may be based on the determining of the first value. Further, the actuating of the second pump may be based on the determining of the at least one second value and the at least one third value.

In further embodiments, the apparatus 1500 may include a gas instilling device and a gas instilling device actuator. Further, the gas instilling device may be fluidly coupled with the interior cavity of the coupling assembly 1512. Further, the gas instilling device may be configured for instilling at least one fourth amount of a therapeutic gas in the interior cavity. Further, the gas instilling device actuator may be operationally coupled with the gas instilling device. Further, the gas instilling device actuator may be configured for actuating the gas instilling device. Further, the instilling of the at least one fourth amount of the therapeutic gas may be based on the actuating of the gas instilling device. Further, in an embodiment, the gas instilling device may be operationally coupled with the first pump. Further, the first pump may be configured for drawing the at least one fourth amount of the air from the interior cavity based on the instilling of the of the at least one fourth amount of the therapeutic gas. Further, the drawing of the at least one fourth amount of the air from the interior cavity maintains the reduced pressure of the first value.

Further, in some embodiment, the varying of the reduced pressure between at least one second value and at least one third value from the first value may be associated with a frequency.

Further, in some embodiment, the varying of the reduced pressure between at least one second value and at least one third value from the first value may be associated with a rate.

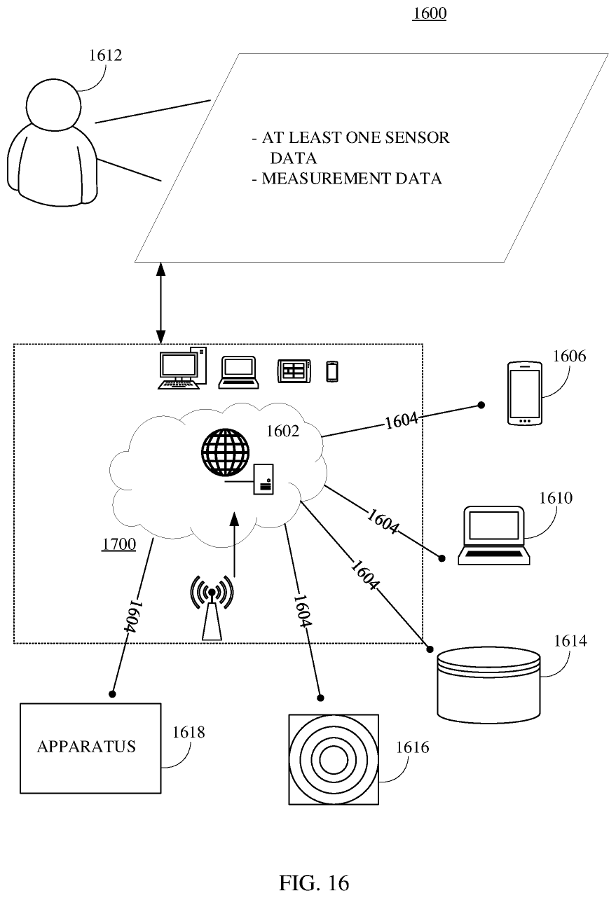

FIG. 16 is an illustration of an online platform 1600 consistent with various embodiments of the present disclosure. By way of non-limiting example, the online platform 1600 to facilitate application of varying pressure to a body of an individual may be hosted on a centralized server 1602, such as, for example, a cloud computing service. The centralized server 1602 may communicate with other network entities, such as, for example, a mobile device 1606 (such as a smartphone, a laptop, a tablet computer etc.), other electronic devices 1610 (such as desktop computers, server computers etc.), databases 1614, sensors 1616, and an apparatus 1618 (such as the apparatus 100, the apparatus 1500, etc.) over a communication network 1604, such as, but not limited to, the Internet. Further, users of the online platform 1600 may include relevant parties such as, but not limited to, end-users, administrators, service providers, service consumers and so on. Accordingly, in some instances, electronic devices operated by the one or more relevant parties may be in communication with the platform.

A user 1612, such as the one or more relevant parties, may access online platform 1600 through a web based software application or browser. The web based software application may be embodied as, for example, but not be limited to, a website, a web application, a desktop application, and a mobile application compatible with a computing device 1700.

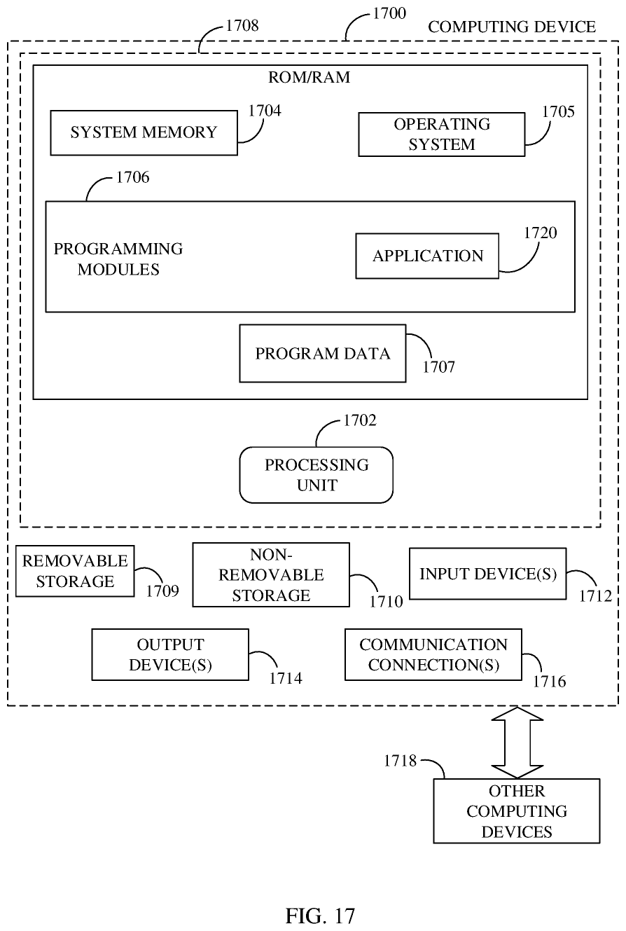

With reference to FIG. 17, a system consistent with an embodiment of the disclosure may include a computing device or cloud service, such as computing device 1700. In a basic configuration, computing device 1700 may include at least one processing unit 1702 and a system memory 1704. Depending on the configuration and type of computing device, system memory 1704 may comprise, but is not limited to, volatile (e.g. random-access memory (RAM)), non-volatile (e.g. read-only memory (ROM)), flash memory, or any combination. System memory 1704 may include operating system 1705, one or more programming modules 1706, and may include a program data 1707. Operating system 1705, for example, may be suitable for controlling computing device 1700's operation. In one embodiment, programming modules 1706 may include image-processing module, machine learning module. Furthermore, embodiments of the disclosure may be practiced in conjunction with a graphics library, other operating systems, or any other application program and is not limited to any particular application or system. This basic configuration is illustrated in FIG. 17 by those components within a dashed line 1708.

Computing device 1700 may have additional features or functionality. For example, computing device 1700 may also include additional data storage devices (removable and/or non-removable) such as, for example, magnetic disks, optical disks, or tape. Such additional storage is illustrated in FIG. 17 by a removable storage 1709 and a non-removable storage 1710. Computer storage media may include volatile and non-volatile, removable and non-removable media implemented in any method or technology for storage of information, such as computer-readable instructions, data structures, program modules, or other data. System memory 1704, removable storage 1709, and non-removable storage 1710 are all computer storage media examples (i.e., memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by computing device 1700. Any such computer storage media may be part of device 1700. Computing device 1700 may also have input device(s) 1712 such as a keyboard, a mouse, a pen, a sound input device, a touch input device, a location sensor, a camera, a biometric sensor, etc. Output device(s) 1714 such as a display, speakers, a printer, etc. may also be included. The aforementioned devices are examples and others may be used.

Computing device 1700 may also contain a communication connection 1716 that may allow device 1700 to communicate with other computing devices 1718, such as over a network in a distributed computing environment, for example, an intranet or the Internet. Communication connection 1716 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer readable media as used herein may include both storage media and communication media.

As stated above, a number of program modules and data files may be stored in system memory 1704, including operating system 1705. While executing on processing unit 1702, programming modules 1706 may perform processes including, for example, one or more stages of methods, algorithms, systems, applications, servers, databases as described above. The aforementioned process is an example, and processing unit 1702 may perform other processes. Other programming modules that may be used in accordance with embodiments of the present disclosure may include machine learning applications.

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, general purpose graphics processor-based systems, multiprocessor systems, microprocessor-based or programmable consumer electronics, application specific integrated circuit-based electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general-purpose computer or in any other circuits or systems.

Embodiments of the disclosure, for example, may be implemented as a computer process (method), a computing system, or as an article of manufacture, such as a computer program product or computer readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random-access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, solid state storage (e.g., USB drive), or a CD-ROM, a carrier wave from the Internet, or other forms of RAM or ROM. Further, the disclosed methods' stages may be modified in any manner, including by reordering stages and/or inserting or deleting stages, without departing from the disclosure.

Although the present disclosure has been explained in relation to its preferred embodiment, it is to be understood that many other possible modifications and variations can be made without departing from the spirit and scope of the disclosure.

What is claimed is:

1. An apparatus for facilitating application of varying pressure to a body of an individual, the apparatus comprising:
a plurality of pumps configured to be fluidly coupled with the body of the individual using a coupling assembly, wherein the coupling assembly is configured to be attached to the body, wherein the coupling assembly comprises an interior cavity, wherein the interior cavity is coupled with a body cavity of the individual, wherein the plurality of pumps comprises a first pump and a second pump, wherein the first pump is configured for drawing a first amount of air from the interior cavity for generating a reduced pressure of a first value in the body cavity, wherein the second pump is configured for drawing at least one second amount of the air from the interior cavity and blowing at least one third amount of the air in the interior cavity for varying the reduced pressure in the body cavity between at least one second value and at least one third value from the first value;
a plurality of actuators comprising a first actuator and a second actuator, wherein the first actuator is operationally coupled with the first pump and the second actuator is operationally coupled with the second pump, wherein the first actuator is configured for actuating the first pump, wherein the second actuator is configured for actuating the second pump, wherein the drawing of the first amount of the air from the interior cavity is based on the actuating of the first pump, wherein the drawing of the at least one second amount of the air from the interior cavity and the blowing of the at least one third amount of the air in the interior cavity is based on the actuating of the second pump; and
at least one valve fluidly coupled with the plurality of pumps, wherein the at least one valve is configured for transitioning between a plurality of valve states based on the reduced pressure, wherein the at least one valve is configured for allowing at least one of entering and exiting of at least one amount of the air from the interior cavity through the at least one valve based on the transitioning, wherein the allowing of the at least one of the entering and the exiting of the at least one amount of the air from the interior cavity maintains the reduced pressure within a predetermined value range of the reduced pressure.

2. The apparatus of claim 1 further comprising an intra oral appliance configured for pushing a soft palate present in a nasal cavity of the body cavity for sealing the nasal cavity from at least one airway associated with the nasal cavity, wherein the drawing of the first amount of the air from the interior cavity is further based on the sealing, wherein the drawing of the at least one second amount of the air from the interior cavity and the blowing of the at least one third amount of the air in the interior cavity is further based on the sealing.

3. The apparatus of claim 2, wherein the intra oral appliance comprises at least one inflatable balloon, wherein the intra oral appliance is configured for inflating the at least one inflatable balloon, wherein the pushing of the soft palate is based on inflating of the at least one inflatable balloon.

4. The apparatus of claim 2, wherein the intra oral appliance comprises at least one tube, wherein the intra oral appliance is configured for blowing a gas through the at least one tube towards the soft palate, wherein the pushing of the soft palate is based on the blowing of the gas through the at least one tube.

5. The apparatus of claim 2, wherein the intra oral appliance comprises at least one electrode, wherein the at least one intra oral appliance is configured for electrically stimulating the soft palate using the at least one electrode, wherein the pushing of the soft palate is based on the electrically stimulating of the soft palate using the at least one electrode.

6. The apparatus of claim 1, wherein the varying of the reduced pressure between at least one second value and at least one third value from the first value is associated with a frequency.

7. The apparatus of claim 6, wherein the frequency varies between a plurality of frequency values of the frequency.

8. The apparatus of claim 1, wherein the varying of the reduced pressure between at least one second value and at least one third value from the first value is associated with a rate.

9. The apparatus of claim 8, wherein the rate varies between a plurality of rate values of the rate.

10. The apparatus of claim 1 further comprising:
a measurement device configured for generating measurement data based on at least one bodily function of the individual; and
a processing device communicatively coupled with the measurement device, wherein the processing device is configured for:
analyzing the measurement data;
determining the first value of the reduced pressure based on the analyzing; and
determining the at least one second value and the at least one third value of the reduced pressure based on the analyzing, wherein the processing device is communicatively coupled with the plurality of actuators, wherein the actuating of the first pump is further based on the determining of the first value, wherein the actuating of the second pump is further based on the determining of the at least one second value and the at least one third value.

11. The apparatus of claim 1 further comprising:
a gas instilling device fluidly coupled with the interior cavity of the coupling assembly, wherein the gas instilling device is configured for instilling at least one fourth amount of a therapeutic gas in the interior cavity; and
a gas instilling device actuator operationally coupled with the gas instilling device, wherein the gas instilling device actuator is configured for actuating the gas instilling device, wherein the instilling of the at least one fourth amount of the therapeutic gas is based on the actuating of the gas instilling device.

12. The apparatus of claim 11, wherein the gas instilling device is operationally coupled with the first pump, wherein the first pump is further configured for drawing the at least one fourth amount of the air from the interior cavity based on the instilling of the of the at least one fourth amount of the therapeutic gas, wherein the drawing of the at least one fourth amount of the air from the interior cavity maintains the reduced pressure of the first value.

13. The apparatus of claim 1 further comprising:
at least one sensor configured for generating at least one sensor data based on a value of a pressure in the body cavity of the individual; and
a processing device communicatively coupled with the at least one sensor, wherein the processing device is configured for:
analyzing the at least one sensor data, wherein the analyzing comprises comparing the value of the pressure with a predetermined value of the pressure; and
generating a command based on the analyzing, wherein the processing device is communicatively coupled with the plurality of actuators, wherein the actuating of the first pump is based on the command.

14. The apparatus of claim 1, wherein the interior cavity is coupled to a body surface of the individual, wherein the drawing of the first amount of the air from the interior cavity generates the reduced pressure on the body surface, wherein the drawing of the at least one second amount of the air from the interior cavity and the blowing of the at least one third amount of the air in the interior cavity varies the reduced pressure on the body surface between at least one second value and at least one third value.

15. An apparatus for facilitating application of varying pressure to a body of an individual, the apparatus comprising:
a plurality of pumps configured to be fluidly coupled with the body of the individual using a coupling assembly, wherein the coupling assembly is configured to be attached to the body, wherein the coupling assembly comprises an interior cavity, wherein the interior cavity is coupled with a body cavity of the individual, wherein the plurality of pumps comprises a first pump and a second pump, wherein the first pump is configured for drawing a first amount of air from the interior cavity for generating a reduced pressure of a first value in the body cavity, wherein the second pump is configured for drawing at least one second amount of the air from the interior cavity and blowing at least one third amount of the air in the interior cavity for varying the reduced pressure in the body cavity between at least one second value and at least one third value from the first value;
a plurality of actuators comprising a first actuator and a second actuator, wherein the first actuator is operationally coupled with the first pump and the second actuator is operationally coupled with the second pump, wherein the first actuator is configured for actuating the first pump, wherein the second actuator is configured for actuating the second pump, wherein the drawing of the first amount of the air from the interior cavity is based on the actuating of the first pump, wherein the drawing of the at least one second amount of the air from the interior cavity and the blowing of the at least one third amount of the air in the interior cavity is based on the actuating of the second pump;
at least one valve fluidly coupled with the plurality of pumps, wherein the at least one valve is configured for transitioning between a plurality of valve states based on the reduced pressure, wherein the at least one valve is configured for allowing at least one of entering and exiting of at least one amount of the air from the interior cavity through the at least one valve based on the transitioning, wherein the allowing of the at least one of the entering and the exiting of the at least one amount of the air from the interior cavity maintains the reduced pressure within a predetermined value range of the reduced pressure;
at least one sensor configured for generating at least one sensor data based on a value of a pressure in the body cavity of the individual; and
a processing device communicatively coupled with the at least one sensor, wherein the processing device is configured for:
analyzing the at least one sensor data, wherein the analyzing comprises comparing the value of the pressure with a predetermined value of the pressure; and
generating a command based on the analyzing, wherein the processing device is communicatively coupled with the plurality of actuators, wherein the actuating of the first pump is based on the command.

16. The apparatus of claim 15 further comprising an intra oral appliance configured for pushing a soft palate present in a nasal cavity of the body cavity for sealing the nasal cavity from at least one airway associated with the nasal cavity, wherein the drawing of the first amount of the air from the interior cavity is further based on the sealing, wherein the drawing of the at least one second amount of the air from the interior cavity and the blowing of the at least one third amount of the air in the interior cavity is further based on the sealing.

17. The apparatus of claim 15 further comprising:
a measurement device configured for generating measurement data based on at least one bodily function of the individual; and
a processing device communicatively coupled with the measurement device, wherein the processing device is configured for:
analyzing the measurement data;
determining the first value of the reduced pressure based on the analyzing; and
determining the at least one second value and the at least one third value of the reduced pressure based on the analyzing, wherein the processing device is communicatively coupled with the plurality of actuators, wherein the actuating of the first pump is further based on the determining of the first value, wherein the actuating of the second pump is further based on the determining of the at least one second value and the at least one third value.

18. The apparatus of claim 15 further comprising:
a gas instilling device fluidly coupled with the interior cavity of the coupling assembly, wherein the gas instilling device is configured for instilling at least one fourth amount of a therapeutic gas in the interior cavity; and
a gas instilling device actuator operationally coupled with the gas instilling device, wherein the gas instilling device actuator is configured for actuating the gas instilling device, wherein the instilling of the at least one fourth amount of the therapeutic gas is based on the actuating of the gas instilling device.

19. The apparatus of claim 15, wherein the varying of the reduced pressure between at least one second value and at least one third value from the first value is associated with a frequency.

20. The apparatus of claim 15, wherein the varying of the reduced pressure between at least one second value and at least one third value from the first value is associated with a rate.
